(19) United States
(12) Patent Application Publication
(10) Pub. No.: US 2005/0148055 A1
(43) Pub. Date: Jul. 7, 2005

(54) Method for the biotechnological production of xylitol

(76) Inventors: Thomas Walther, Dresden (DE); Kai Ostermann, Dresden (DE); Hans-Frieder Listewnik, Leipzig (DE); Thomas Blev, Leipzig (DE); Gerhard Roedel, Karlsfeld (DE)

Correspondence Address: Law Offices of Karl Hormann, PO Box 381516, Cambridge, MA, 02238-1516, US

(21) Appl. No.: 10/514,324

(22) PCT Filed: May 15, 2003
(86) PCT No.: PCT/DE03/01619

(30) Foreign Application Priority Data
May 15, 2002 (DE) 102 22 373.4

(51) Int. Cl. C12P 7/18; C12N 1/18

(52) U.S. Cl. 435/158; 435/254.2; 435/254.21; 435/254.23

(57) ABSTRACT

The invention relates to a method for the biotechnological production of xylitol, in which micro-organisms capable of metabolizing xylose to xylitol are used by modifying micro-organisms such that oxidation of NADH by enzymes other than the xylose reductase is reduced or excluded, cultivating the micro-organisms in a substrate containing xylose and 10-40 grams per liter of sulfite salt, and enriching the xylitol and recovering it from the substrate.

### BACKGROUND OF THE INVENTION

1. Field of the Invention

The invention relates to a method of biologically producing xylitol by applying micro-organisms capable of metabolizing xylose into xylitol

Xylitol is a sugar alcohol occurring in nature and is used primarily as a sugar surrogate in dietetic food stuffs as in the metabolism it breaks down independently of insulin. Xylitol is also used by the pharmaceutical industry, for instance, for tooth paste.

A large field of application of xylitol resides in chewing gum, chewing tablets and similar products since its sweetening strength is approximately equal to that of sucrose without its carious action, however.

2. The Prior Art

According to the prior art, xylitol is chemically synthesized by reducing xylose with nickel catalysts under high pressure and at high temperatures with an average yield of 50 to 60% of the xylose used. Xylose is a major component of vegetable raw materials and is produced by acidic hydrolysis or sulfite leching of hemicellulose-rich vegetable scraps.

With a view to overcoming the disadvantage of low xylitol yields in chemical processes, EP 0,716,067 is proposing a multiple-step chemical process to produce xylitol, proceeding from gluconic acid, to obtain yields between 60 and 70%.

The chemical processes have in common the use of cost-intensive or environmentally hazardous catalysts required for the chemical production of xylitol. Nickel catalysts, the use of which is questionable for ecological reasons, of are used primarily.

Furthermore, these chemical processes operate at least in part at high temperatures between 70 and 150° C. as well as at high pressures of up to 10 MPa. In order to obtain the xylitol in a purified state, complex cleaning and concentration steps are also required.

As an alternative to the chemical production of xylitol, the prior art refers to high-yield biotechnological processes of metabolizing xylose to xylitol.

DE 44 10 028 A1 discloses maintaining yeasts under special culture conditions for increasing the yield of xylitol. In this connection, the pH-value of the culture medium is maintained in a particular range in order to stimulate the yeasts to a heightened xylitol production.

The micro-organisms used are yeasts of the species candida, debaryomyces and pichia which use xylose naturally and which metabolize xylose to xylitol under limited oxygen. The reaction is catalyzed by the xylose reductase (XR) enzyme, aided by the cofactor NADH.

As an alternative to natural xylitol producers, micro-organisms are gene-technologically modified in accordance with the teaching of EP 0,672,161 such that after manipulation they are capable of forming xylitol from other sources of carbon.

Another approach to increasing the production of xylitol biologically is described in EP 0,604,429. In that case, xylitol synthesizing and metabolizing yeasts are gene-technologically modified such that the enzymes which convert the desired end-product are cut off.

In accordance with the teaching of this invention, the expression of those genes which lead to a conversion or reduction of the desired xylitol end-product is cut off or reduced.

Examples to be mentioned from the prior art are the publications in Chemical Abstracts 129:25507; 131-4270; 133:236915 as well as 125:270220. Here, micro-organisms are genetically manipulated with only individual aspects of the metabolistic system being deliberately and successfully altered; but no attention is paid to the complexity of the metabolistic system of the micro-organisms. Such approaches regularly result in individual aspects of the alterations being successfully realized; but the micro-organism itself suffers limitations, e.g. in its reproductive ability, in consequence of these interventions.

Also, processes are known in the prior art in which micro-organisms which do not naturally reduce xylitol are gene-technologically enabled to form xylitol reductase enzymes and which can thus be used for the production of xylitol.

It is also known strongly to over-express this xylitol reductase enzyme.

The mentioned bio-technological processes commonly share that even at a strong over-expression of xylitol reductase or by cutting-off the enzymes participating in the natural metabolism of xylitol, the production of xylitol cannot be increased sufficiently effective to produce xylitol bio-technologically in a cost-efficient manner.

### OBJECT OF THE INVENTION

It is thus an object of the invention to provide a method by which a higher production of xylitol from xylose is made possible bio-technologically and which provides for a more economical process operation.

### SUMMARY OF THE INVENTION

The object is accomplished by a process of bio-technologically producing xylitol wherein micro-organisms are being used which can metabolize xylose into xylitol and which includes the following process steps:

a) modifying micro-organisms such that the oxidation of NADH by enzymes other than xylitol reductase is reduced or avoided;

b) cultivating the micro-organisms in a substrate containing xylose and 10 to 40 grams per liter of a sulfite salt in a aerobic growth phase and a limited oxygen xylitol production phase; and

c) enrichment and recovery of xylitol from the substrate.

The conversion of xylose to xylitol takes place as follows:
xylose+NADH_{2→}^{+XR}xylitol+NAD⁺

As has already be described, this reaction is catalyzed by the xylose reductase enzyme with participation of the cofactor NADH. As has been set forth in the description of the prior art relating to this field, an excess of the xylose reductase enzyme which catalyzes the reaction by an over-expression of the enzyme with modified micro-organisms does not result in a significant increase and, therefore, economic improvement of xylitol production.

It has surprisingly been found that the reaction of xylose to xylitol is strongly dependent upon the availability of the cofactor NADH and that it is limited by it.

In accordance with the invention, the micro-organisms are modified such that other NADH consuming enzyme systems of the micro-organisms, such as, for instance, mitochondrial NADH-dehydrogenase, alcohol-dehydrogenase and glycerol phosphate-dehydrogenase are cut off.

The effect of the modification of the micro-organisms in accordance with the invention is based upon the fact that the mentioned enzymes have a higher affinity to this cofactor.

If, preferably, the NADH is oxidized to NAD(P) by the enzymes competing with the xylose reductase, the required cofactor will not be available in the quantity which is necessary for a high production yield of xylitol.

By the inventive use of genetically modified micro-organisms it is possible by the process in accordance with the invention to obtain significantly increased yields of xylitol. Accordingly, by overcoming the described disadvantages of other bio-technological processes, the instant process is an attractive alternative to prior art chemical processes.

In addition, it is a particular advantage of the process in accordance with the invention that it uses renewable raw materials and, by contrast with conventional chemical processes utilizing nickel catalysts, that it results in no environmentally hazardous effects.

Moreover, it is inherent in the bio-technological processes that they operate at comparatively mild reaction conditions, and that in general they are environmentally friendlier than chemical processes. The description of embodiments demonstrates how individual micro-organisms, yeasts in particular, are initially modified and subsequently used for producing xylitol.

Yeasts of the species saccharomyces are of particular significance in connection with bio-technological processes. With these yeasts, NADH is oxidized in various metabolistic ways. The primary ones to be mentioned are alcohol fermentation, glycerol production and the oxidation by the external mitochondrial NADH dehydrogenase. These enzymes have a higher affinity to NADH compared to xylose reductase, and the cofactor is thus preferably oxidized by these enzymes.

As a result of the consumption of NADH by the sketched break-down ways the availability of NADH for the xylose reduction is limited, and xylitol cannot be produced in greater quantities.

In accordance with the invention the amount NADH available in the cytoplasm for reaction with the xylose reductase is increased by rendering inactive, or suppression the expression of, one or more enzymes which cause the NADH to oxidize.

The internal mitochondria membrane of the yeasts is impervious for NADH; hence the modification of the enzyme system is directed towards the cytoplasmic NADH dehydrogenases responsible for the oxidation.

In saccharomyces cerevisiae genes NDE1 and NDE2 code the external NADH dehydrogenases, whereas GPD1 and GPD2 code the glycerol phosphate dehydrogenases. The suppression of one or more of the mentioned genes thus result, in accordance with the invention, in a reduced NADH consumption by the alternative ways of metabolism, and the NADH is available for xylose reductase to a greater extent.

In accordance with a preferred embodiment of the invention, the break-down of glucose into ethanol is also inhibited in saccharomyces cerevisiae by the addition of sulfite salt (e.g. calcium hydrogen sulfite, sodium sulfite, potassium sulfite) at a concentration of 10 to 40 grams per liter of substrate. Consequently, glycerol production is increased.

In accordance with the invention, the formation of glycerol is counteracted by gene-technologically suppressing the expression of the NADH-consuming glycerol phosphate dehydrogenase enzyme.

Under these conditions, xylitol production is increased by using the NADH cofactor from the xylose reductase made available by the suppression of the glycerol phosphate dehydrogenase for producing xylitol from xylose.

The effect of the inventive process with modified micro-organisms is the result, in one embodiment, by changing the genes of the micro-organisms by recombinant DNA technology. Alternatively, the modification of the micro-organisms may be carried out by deliberate or accidental mutagenesis.

The modification of the micro-organisms is carried out such that in a first step those sequences of the enzymes which use the NADH factor are destroyed or manipulated such that they cannot by produced by the micro-organism and can only be synthesized with reduced activity.

Such methods of gene-technologically modifying micro-organisms are well known in the prior art. The micro-organisms manipulated in this manner produce more xylitol and accumulate it in the growth medium from which the xylitol may be enriched or recovered by crystallization or by chromatographic processes. The ensuing description of embodiments explains the procedure of modifying the micro-organisms and of recovery of xylitol.

### DETAILED DESCRIPTION OF THE PREFERRED EMBODIMENTS

### EXAMPLE 1

Initially, the production of yeast strains which express the heterologous xylose reductase will be described.

The expression of heterologous xylose reductase in yeast is well known in the prior art and has been described in patent WO 91/15588.

Pichia stipitis was cultivated overnight in 100 ml YPD (1 l of medium contains 10 g of yeast extract, 20 g of peptone and 20 g of glucose, pH=6.5) at 30° C. Cells from 10 ml of cultivation medium were pelletized by centrifuging, and the chromosomal DNA was isolated in accordance with the protocol according to Kaiser et al. (1994). The DNA served as a template for PCR amplification of the 956 Bp sized intronless open reading frame (ORF) of the XYL1-gene (Amore et al. 1993). The resultant fragment was separated in a 1% agaro sail and purified with the Genomed company “JETQUICK Gel Extraction Spin Kit”. The plasmid was digested with the corresponding restriction endonucleases and purified in the manner described above. The vector used for the expression in S. cerevisiae was p425GPD. The vector is a multi-copy plasmid and contains the LEU2-gene of S. cerevisiae as a selection marker (Mumberg et al. 1995). The vector was digested with the same restriction enzymes as the fragment and was appropriately purified. The ORF of the XYL1-gene was ligated into the vector, and recombinant plasmids were identified by plasmid preparation and restriction analyses. In the plasmid thus produced (p425GPD-PsXYL1) the transcription of XYL1 is controlled by the strong GPD promoter and ensures a high expression of exogenic XR in S. cerevisiae. The strain S. cerevisiae KOY50 (MATa; his3Δ1; leu2Δ0; ura3Δ0) was transformed with p425GPD-PsYXL1 in accordance with the method of Schiestl and Gietz (1989). Leucine-prototrophic transformands were isolated and analyzed. the controls from the Genomed company.

### EXAMPLE 2

Inactivation of the NADH dehydrogenases by gene-replacement of the genes NDE1 and NDE2.

The mitochondrial localized cytosolic NADH dehydrogenase competes with the XR for the cofactor NADH. In order to reduce the consumption of cytosolic NADH, the NDE1 and NDE2 genes were inactivated by replacement with suitable DNA-fragments. A replacement fragment of 40 bp homology directly upstream (5′ side) of the start condon of NDE1 and 40 bp homology downstream (3′ side) from the stop condon was amplified by PCR. The fragment contains the his5⁺-gene of schizosaccharomyces pombe and complements his3 mutations in S. cerevisiae (Wach et al. 1997). After amplification the fragment was, as describes above, isolated and purified. S. cerevisiae was transformed with the replacement fragment according to the method of Schiestl and Gietz (1989). Histidine-prototrophic transformands were isolated. The correct integration of the fragment and the exchange of NDE1 was proven by diagnostic PCR. Such a strain (KOY50Δnde1) was transformed with p425GPD-PsXYL1, as described in Example 1, and used for the production of Xylitol.

A replacement fragment with homologies to NDE2, as described above, was constructed for the production of the double-mutant nde1/nde2. The selection marker was the kanMX module (Wach et al. 1994). This module renders transformands resistant against G418 (genticine). After the transformation, the KOY50Δnde1 cells were plated onto a medium containing G418. Resistant clones were examined by diagnostic PCR for the correct insertion of the replacement fragment and inactivation of NDE2. The resultant strain (KOY50Δnde1Δnde2) was transformed with p425GPD-PsXYL1, as described in Example 1, and used for xylitol production.

### EXAMPLE 3

Gene-replacement of the genes GPD1 and GPD2 coding glycerol phosphate dehydrogenase.

The cytoplasmic glycerol phosphate dehydrogenases use NADH as cofactor. Their inactivation results in preferential oxidation of cytosolic NADH by the reduction of xylose to xylitol by means of XR. The strains S. cerevisiae KOY50Δnde1 or KOY50Δnde1Δnde2 were used for the gene replacement.

Both strains carry the ura3Δ0 mutation. Fragments with homologies to GPD1 and GPD2 as described under Example 2, were used for the disruption. The fragments contained the URA3-gene of candida albicans as selection marker. The selection marker was flanked by DNA sections identical to each other (Goldstein et al. 1999). URA3 from c. albicans complements ura3 mutations in s. cerevisiae. Cells with an intact uracil-metabolism are, in contrast to cells with a ura3 mutation, sensitive to 5-fluoro-orotic acid 3 (5-FOA). After integration of the disruption cassette into the genome spontaneous homologous recombination takes place at the repeating DNA-sections, and this causes loss of the URA3-marker. Clones in which such a recombination has occurred can be easily isolated by their resistance to 5-FOA.

The DNA-fragments described above were amplified by PCR and purified. The fragment for the gene-replacement of GPD1 was transformed in s. cerevisiae KOY50Δnde1 or KOY50Δnde2, respectively, in accordance with the method of Schiestl and Gietz (1989). Uracil-prototrophic transformands were isolated on the corresponding selection medium. The replacement of GDP1 was confirmed by diagnostic PCR. Thereafter, the strain thus constructed were selected for resistance against 5-TOA. In 5-FOA resistant strains, the loss of the URA3-marker was proven by diagnostic PCR. In further work, GPD2 was inactivated, as described above.

The strains thus constructed were transformed with p425GPD-PsXYL1, as described in Example 1, and used for producing xylitol.

### EXAMPLE 4

Production of strains with inactivated dehydrogenases by tetrad analysis.

In order to produce strains with several inactivated dehydrogenases, the corresponding cells were initially inactivated individually, as described in Example 2. Tetrads were analyzed after crossing of the individual mutants and sporulation of the produced diploid strains. Double mutants can be identified in a simple manner in the non-parental ditype (NPD). The KOY50 strain was used individually to inactivate GPD1 and GPD2 by gene-replacement, as described for NDE1 in Example 2. Transformands of opposite pairing type were crossed and the resultant strains were brought to sporulate on the corresponding medium. The tetrads were tested for histidine-prototrophy. The GPD1/GPD2 double zero mutants were isolated in NPD-tetrads as histidine-prototrophic single spore clones, transformed with p425GPD-PsXYL1 and used for the production of xylitol.

1. (canceled)

2. The method according to claim 7, characterized by the fact that the modified micro-organisms have a reduced or no activity of at least one of the glycerol phosphate-dehydrogenase, external mitochondrial NADH-dehydrogenase or alcohol-dehydrogenase enzymes.

3. The method according to claim 2, characterized by the fact that in the micro-organisms the expression of those genes which code the glycerol phosphate-dehydrogenase, external mitochondrial NADH-dehydrogenase or alcohol-dehydrogenase are selected or reduced.

4. The method according to claim 3, characterized by the fact that the micro-organisms used are yeasts of the species candida saccharomyces, pichia, kluyveromyces, schizosaccharomyces and debaryomyces.

5. The method according to claim 4, characterized by the fact that in saccharomyces cerevisiae at least one of the NDE1, NDE2, GPD1 and GPD2 genes is removed or inactivated.

6. The method according to claim 5, characterized by the fact that in the substrate galactose, mannose, glucose or arabinose is used as a further source of carbon.

7. A method of biotechnologically producing xylitol by the use of micro-organisms capable of metabolizing xylose into xylitol, whereby the method comprises the following steps:
a) modifying the micro-organisms such that the oxidation of NADH is reduced or prevented by enzymes other than xylose reductase;
b) cultivating the micro-organisms in a substrate containing xylose and 10-40 grams per liter of a sulfite salt in an aerobic growth phase and a limited oxygen xylitol production phase; and
c) enriching and recovering the xylitol from the substrate.

8. The method of claim 7, wherein the sulfite salt used is selected from the group consisting of calcium hydrogen sulfite, sodium sulfite and potassium sulfite.
